(19) United States
(12) Patent Application Publication
(10) Pub. No.: US 2023/0048678 A1
(43) Pub. Date: Feb. 16, 2023

(54) ICAN DERIVATIVES FOR ANTIFUNGAL USE

(71) Applicant: DEBRECENI EGYETEM, Debrecen (HU)

(72) Inventors: Sándor KÉKI, Debrecen (HU); Alexandra KISS, Mándok (HU); László MAJOROS, Debrecen (HU); Miklós NAGY, Hajdúnánás (HU); Zsolt László NAGY, Szeghalom (HU); István PÓCSI, Debrecen (HU); Dávid RÁCZ, Debrecen (HU); Gábor SZEMÁN-NAGY, Debrecen (HU); Zsuzsa MÁTHÉNÉ SZIGETI, Debrecen (HU); László TÁLAS, Bocskaikert (HU); Zoltán TÓTH, Debrecen (HU); Miklós ZSUGA, Debrecen (HU)

(21) Appl. No.: 17/788,284

(22) PCT Filed: Dec. 23, 2020
(86) PCT No.: PCT/HU2020/050064

§ 371 Date: Jun. 22, 2022

(30) Foreign Application Priority Data
Dec. 23, 2019 (HU) P1900454

(51) Int. Cl. A61K 31/277; A01N 37/34; A01P 3/00; A61P 31/10

(52) U.S. Cl. CPC A61K 31/277; A01N 37/34; A01P 3/00; A61P 31/10

(57) ABSTRACT

The invention relates to ICAN-derivative compounds having the general formula (I), wherein Q is selected from the group consisting of —NC, —NO₂, —NO, —NHOH, —NCS and —NR₁R₂, for use as antifungal agents and gal preparations and pharmaceutical preparations for use in the treatment of a subject in need of antifungal medication.

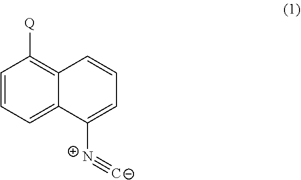

### FIELD OF THE INVENTION

The invention relates to ICAN-derivative compounds as defined herein, their use as antifungal agents and any further antifungal uses, pharmaceutical and non-pharmaceutical antifungal preparations as well as said ICAN-derivative compounds and pharmaceutical preparations for use in the treatment of a subject in need of antifungal medication.

### BACKGROUND ART

Invasive fungal infections cause 1.5 million deaths annually and may show a further increase in the following decades. Most of these infections are caused by Candida species, followed by Aspergillus and Cryptococcus neoformans [Pfaller M A et al., 2007, Bongomin F et al., 2017, Garcia-Solache M A et al., 2017].

Members of Candida genus are part of the healthy human microbiome and can be found in oral cavity, gastrointestinal and genital tract, however as opportunistic pathogens, these yeasts can cause a wide variety of diseases ranging from superficial infections to life threatening invasive candidiasis [Bondaryk M et al., 2013, Odds F C et al., 2007, Cauchie M et al., 2017]. Incidence and mortality of the latter remained high in the last decades, despite the widespread use of echinocandins and newer generation triazoles as prophylactic and therapeutic agents [Lortholary O et al., 2014, Hirano R et al., 2015]. Development of invasive candidiasis is associated with several predisposing factors, notably with immunosuppression, recent abdominal surgery, diabetes, broad spectrum antibiotic therapy and many others [Yapar N et al., 2014]. While risk factors are numerous, therapeutic options are very limited with only three major antifungal classes (triazoles, polyenes, echinocandins) available [Ben-Ami R., 2018]. The most recent antifungal compounds, the echinocandins were introduced almost twenty years ago and apparently there are few new drugs to come in the following years [Perfect J R 2017].

The development of new antifungal compounds is constrained because fungi are eukaryotic cellular targets, which, if disrupted, can also damage host tissues. The increase in fungal infections and increase in use of antifungal compositions has resulted in emergence of resistance among fungi. Anti-fungal resistance has high clinical impact as fungal diseases are causing an increase in morbidity and mortality of immunocompromised patients.

Although antifungal treatment options are currently available for mycoses including systemic mycoses and an effective treatment can be found against most fungal pathogens, they present limitations such as toxicity, drug-drug interactions, and emergence of clinical resistance [Souza ACO, 2017].

Moreover, the emergence of new, drug resistant pathogenic fungi, such as Candida auris therefore poses a serious therapeutic challenge and highlights the need for new compounds with different mechanisms of action [Cortegiani A et al., 2018, Krysan D J, 2016].

Kouznetsov V. V. et al. [Kouznetsov V V et al. 2012] disclose cytotoxic and antifungal activities of diverse α-naphthylamine derivatives which are, however, chemically different from those of the present invention as they do not comprise the cyano group which proved to be essential in the present invention.

Recently, in the laboratory of the present inventors a novel amino-isocyanonaphthalene (ICAN) based solvatochromic fluorophore family [Racz D et al., 2018] has been developed. A number of amino-isocyanonaphtalene derivative compounds have been disclosed as fluorophores already in WO2014/184762A1 (Zsuga et al.). The compounds have found numerous and versatile use in both analytical chemistry [Nagy M et al., 2018a, Nagy M et al., 2019, Nagy M et al., 2014] and cell-biology [Nagy M et al., 2016, Nagy M et al. 2018b, Nagy Z L et al. 2018] but no medical effect has been described.

Drobnica L. et al. [Drobnica L 1968] have investigated antifungal activity of aromatic isothiocyanate derivatives and have found that 1-naphthyl- and 2-naphthylisothiocyanates had antifungal effect. The biological activity of said compounds were tested on three types of fungus. The most active compounds (with the lowest ED50 value) were the 1- and 2-naphthyl-isothiocyanates., however, the compounds of Drobnica et al do not comprise cyano groups and other substitutents of the present invention. In particular, the most effective compounds of D1 have a single isothiocyano-group only, and are not substituted with an isocyano-group in position 5 of the naphthyl-ring neither with an amino-group or a group derived from an amino group in position 1.

The present inventors have unexpectedly observed a strong antimycotic effect of the compounds of the present invention and found that the compounds can be used against various fungi including pathological fungi. Thus the invention provides a novel antifungal compound family, which can be used in several fields of application or instead of approved antifungal drug classes. In particular, 1-N-methylamino-5-isocyano-naphthalene (MICAN) was found effective on a commercial strain of Saccharomyces cerevisiae. Subsequently further experiments have been designed to discover whether the ICAN derivatives could be used as potential drugs against pathological fungi and tested the antifungal effect of 1-amino-5-isocyanonaphthalene and its derivatives on different Candida species in vitro and the most effective agent in vivo has been studied in a murine model of invasive candidiasis. Thus, the invention provides a novel antifungal compound family, which can be used in several fields of application or instead of approved drug classes.

The aim of the application is to provide new antifungal compounds for alternative ways of therapy.

The differences between Di—which is considered to be the closest prior art—and the present application are as follows: the most effective compounds of DI bear only one isothiocyano-group, while the present compounds are substituted with an isocyano-group in position 5 of the naphthyl-ring and with an amino-group or a group derived from that in position 1. The biological tests demonstrated in the description support the fact, that the present compounds have similar or even higher activity against fungus than the well-known antifungal compound Amphotericin B.

### BRIEF DESCRIPTION OF THE INVENTION

The invention relates to a compound as defined herein for use in the treatment of a subject in need of antifungal medication.

The invention relates to a compound having general formula (I)

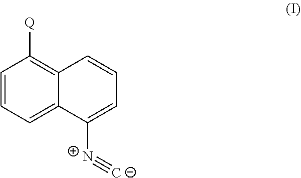

wherein Q is selected from the group consisting of
cyano (—NC), nitro (—NO2), nitroso (—NO), hydroxylamino (—NHOH), isothiocyano (—NCS), or an amino having the general formula (i):

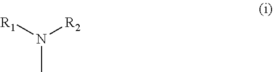

wherein
R₁ is H or a C₁-C₄ alkyl, a C₂-C₄ alkenyl or a C₂-C₄ alkynyl,
R₂ is H or a C₁-C₄ alkyl, a C₂-C₄ alkenyl or a C₂-C₄ alkynyl,
or R₁ and R₂ together form a C₃-C₈ cykloalkyl or a C₅-C₈ cykloalkenyl,
   or a pharmaceutically acceptable salt thereof,
   for use as an antifungal agent in the treatment of a subject in need thereof, preferably of a subject having a fungal disease caused by a fungal pathogen.

The invention also relates to any novel compound as defined above (i.e. without limitation to a medical indication) with the proviso that said compound is different from the following compounds: 1-amino-5-isocyanonaphthalene (ICAN), 1-N-methylamino-5-isocyanonaphthalene (MICAN) and 1-N,N-dimethylamino-5-isocyanonaphthalene (DIMICAN).

In a preferred embodiment said compound has formula (II)

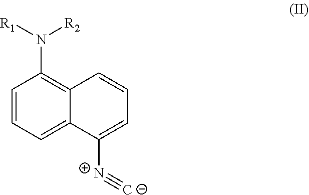

wherein in the formula
R₁ is H or a C₁-C₄ alkyl, a C₂-C₄ alkenyl or a C₂-C₄ alkynyl, AND
R₂ is a C₁-C₄ alkyl, a C₂-C₄ alkenyl or a C₂-C₄ alkynyl,
or R₁ and R₂ together form a C₃-C₈ cykloalkyl or a C₅-C₈ cykloalkenyl

### OR

R₁ is H or a C₁-C₃ alkyl, a C₂-C₃ alkenyl or a C₂-C₃ alkynyl, AND
R₂ is a C₁-C₄ alkyl, a C₂-C₄ alkenyl or a C₂-C₄ alkynyl,
or R₁ and R₂ together form a C₄-C₇ cykloalkyl or a C₅-C₇ cykloalkenyl

### OR

R1 is H or a C₁-C₄ alkyl, a C₂-C₄ alkenyl or a C₂-C₄ alkynyl, AND
R₂ is a C₁-C₃ alkyl, a C₂-C₃ alkenyl or a C₂-C₃ alkynyl,
or R₁ and R₂ together form a C₄-C₇ cykloalkyl or a C₅-C₇ cykloalkenyl;
   in a preferred embodiment,
R₁ is selected from the group consisting of H, methyl, ethyl and propyl,
R₂ is selected from the group consisting of methyl, ethyl and propyl,
or R₁ and R₂ together form a cyclobutyl, cyclopentyl, cyclohexyl or cycloheptyl.

### OR

R₁ and R₂ is, independently, selected from the group consisting of H, methyl, ethyl and propyl, with the proviso that at least one of R₁ and R₂ is different from H,
or R₁ and R₂ together form a cyclobutyl, cyclopentyl, cyclohexyl or cycloheptyl.
   for use as an antifungal agent in the treatment of a subject in need thereof, preferably of a subject having a fungal disease caused by a fungal pathogen.

The invention also relates to any novel compound as defined above (i.e. without limitation to a medical indication) with the proviso that said compound is different from the following compounds: 1-amino-5-isocyanonaphthalene (ICAN), 1-N-methylamino-5-isocyanonaphthalene (MICAN) and 1-N,N-dimethylamino-5-isocyanonaphthalene (DIMICAN).

In a preferred embodiment the invention relates to a novel compound selected from N-ethyl-1-amino-5-isocyanonaphthalene (EICAN) N,N-diethyl-1-amino-5-isocyanonaphthalene (DIEICAN) N-(prop-1-yl)-1-amino-5-isocyanonaphthalene (PICAN) and N,N-dipropyl-1-amino-5-isocyanonaphthalene (DIPICAN).

In a preferred embodiment the compound for use according to the invention is selected from the group consisting of

1-amino-5-isocyanonaphthalene (ICAN)

N-methyl-1-amino-5-isocyanonaphthalene (MICAN)

N-ethyl-1 -amino-5-isocyanonaphthalene (EICAN)

N-(prop-1-yl)-1-amino-5-isocyanonaphthalene (PICAN)

N,N-dimethyl-1-amino-5-isocyanonaphthalene (DIMICAN)

1,5-diisocyanonaphthalene (DIN),

and optionally

N,N-diethyl-1-amino-5-isocyanonaphthalene (DIEICAN) and

N,N-dipropyl-1-amino-5-isocyanonaphthalene (DIPICAN).

In a preferred embodiment the compounds of the invention or the compounds for use of the invention show antifungal effect in an antifungal susceptibility test as described herein.

In a preferred embodiment the invention relates to compositions comprising said novel compounds as defined above. Preferably the invention relates to an anti-fungal use of said composition as defined herein.

In a preferred embodiment the invention relates to pharmaceutical compositions comprising any compounds as defined above preferably for therapeutic antifungal use as defined herein.

In a preferred embodiment the invention relates to a compound for use according to the invention, or the use of a composition of the invention against a fungus, i.e. against a fungal target wherein the fungus is a pathogenic species from any of the following genera: Candida, Aspergillus, Alternaria, Fusarium, Cryptococcus, Histoplasma, Pneumocystis, Stachybotrys, Batrachochytrium and Trichosporon, preferably Candida, Aspergillus Cryptococcus and Trichosporon.

Particularly preferred fungal targets include pathogenic Candida species, such as C. albicans, Candida glabrata, Candida lusitaneae, Candida parapsilosis, Candida tropicalis, Candida krusei and Candida dubliniensis, pathogenic Aspergillus species, such as A. fumigatus, Aspergillus niger, Aspergillus flavus and Aspergillus clavatus, pathogenic Cryptococcus species, such as Cryptococcus neoformans, Cryptococcus laurentii, Cryptococcus albidus and Cryptococcus gattii, and pathogenic Trichosporon species, such as Trichosporon ovoides, Trichosporon inkin, Trichosporon asahii, Trichosporon mu-coides, Trichosporon asteroides, and Trichosporon cutaneum {all previously considered under the general name of Trichosporon beigelii), and Trichosporon dermatis, Trichosporon dohaense and Trichosporon loubieri.

In a preferred embodiment the fungal pathogen belongs to division Ascomycota, in particular to yeasts or true yeasts (Saccharomycotina) within the kingdom of fungi, more preferably to class Sacharomycetes including the family Saccharomycetaceae comprising among others Candida.

In a highly preferred embodiment the fungal pathogen is Candida and/or the fungal disease is candidiasis.

In an embodiment of the invention provides a pharmaceutical composition or a medicament. In particular the invention provides a pharmaceutical composition for use in a method of preventing or treating a fungal disease as disclosed herein (e.g. against a pathogenic fungus as listed herein), preferably a fungal infection in or on a human or animal subject by a fungal pathogen, said composition comprising the compound for use according to the invention, or an antifungal compound as defined herein or in the appended claims, and a pharmaceutically acceptable excipient, carrier or diluent. The animal is preferably a vertebrate animal, e.g. a fish, amphibian, reptilian, bird or mammal, in particular a mammal.

The composition contains at least one anti-fungal compound of the invention and a pharmaceutically-acceptable carrier. In the composition, the anti-fungal compound of the invention is present in an amount effective to treat a fungal infection.

The fungal disease preferably the infection may affect several parts of the human or animal body. In a typical embodiment, the fungal infection relates to an organ of the body which provides barrier function, like the skin, respiratory tract, gastrointestinal tract or the genital-urinary tract.

Preferably the fungal infection or mycosis of the subject may be selected from the group consisting of superficial mycoses, cutaneous mycoses, subcutaneous mycoses and systemic mycoses including systemic mycoses due to primary pathogens and systemic mycoses due to opportunistic pathogens. In a particular embodiment systemic mycoses due to opportunistic pathogens is selected from the group consisting of infections of patients with immune deficiencies and immunocompromised conditions, e.g. AIDS, inherited or acquired disorders of granulocytes, inherited or acquired disorders of T lymphocytes, primary immunodeficiencies, alteration of normal flora by antibiotics and patients with immunosuppressive therapy, patients with anti-cancer therapy like chemotherapy, radiation therapy and therapy against metastatic cancer. Examples of opportunistic mycoses include Candidiasis, Cryptococcosis and Aspergillosis.

Pharmaceutical compositions including medicaments selected from the group consisting of topical preparations e.g. preparations by transdermal delivery, in particular in case of superficial, cutaneous and subcutaneous infections, inhalable preparations, oral preparations, including solid preparations and oral washes, transmucosal preparations including nasal, sublingual, vaginal or rectal delivery preparations and injections, such as intradermal, subcutaneous, intramuscular, intravenous, and intraperitoneal injections.

Any of the compounds or compositions preferably the preferred ones, of the invention as defined above is used against the fungal infection or mycoses as disclosed herein, as defined above.

### Method of Treatment

The invention also relates to a method for treating the fungal infections in a human or animal subject, as taught herein, caused by fungal pathogens, said method comprising administration of the compound or pharmaceutical composition or preparation of the invention in an amount sufficient to reduce the level of the fungal pathogen in the subject or to prevent proliferation or suppressing growth thereof or to eliminate the fungal pathogen. Reducing the level also involves reducing the number of fungal cells or reducing the amount of the fungal pathogen in the subject, including reducing the number or amount in a given tissue or organ or body area.

The invention also relates to a prophylactic treatment of a human or animal subject, comprising administering to said subject the compound or the pharmaceutical preparation or formulation of the invention in an amount sufficient to prevent proliferation of the fungal pathogen or to control the fungal pathogen or to lower the level of the fungal pathogen.

Any of the compounds of the invention as defined above is used against the fungal infection or mycoses as disclosed herein or in the method of treatment, preferably the preferred compounds as defined above.

The invention also relates to a use of the compound of the invention as an antifungal agent.

In an embodiment the antifungal compound of the invention is used as a disinfectant to prevent the growth of and/or to kill a fungus e.g. a pathogenic fungus or a harmful or noxious fungus on an article that is to be ingested by, or placed directly on or in, a human or animal, or a surface that is in need thereof {e.g. a surface that may, directly or indirectly, come into contact with a human or animal) so that there is a risk of a human or animal becoming infected with said pathogenic fungus.

In a preferred embodiment the compound, i.e. the antifungal agent of the invention is used within or on a foodstuff to prevent the growth of a human/animal pathogenic fungus on or within that foodstuff. By foodstuff it is intended to mean any liquid or solid substance which is edible or potable or is intended for consumption for nutritional or pleasurable reasons.

In an embodiment the compound of the invention is used as an antifungal preservative.

In an embodiment the antifungal compound of the invention is used to control fungi. Preferably said antifungal compound is used as a fungicide in a method which is different from a method for the treatment of human or animal body. In another embodiment the invention relates to a composition against fungal pathogens of plants. In a preferred embodiment the compound of the invention is used as a fungicide in or on a plant, for example in or on a plant of agricultural or horticultural importance, e.g. for plant protection against fungal infection.

The invention also relates to an antifungal use of the compounds of the invention or compounds as disclosed herein or compositions as disclosed herein which is a non-medical use.

Any of the compounds of the invention as defined above is used against the non-medical use as disclosed herein or in the compositions, preferably the preferred compounds as defined above.

In an alternative preferred embodiment the compound of the invention is used within or on a medical device or instrument. In an embodiment a method of the invention comprises the step of contacting the substrate with an effective amount of the anti-fungal compound of the invention.

The composition may have a preferred formulation of a liquid, spray, gel, powder, paint etc.

In a further alternative or preferred embodiment the compound of the invention is used on a surface that is contaminated or assumed to be contaminated or expectably would be contaminated by a fungus or a fungal contaminant or a fungal pathogen. The surface to which the compound i.e. the antifungal agent of the invention may be applied may be located within an environment where the presence of fungal infection is possible and/or is to be prevented or controlled, e.g. wherein:

(a) medical examination, diagnosis or treatment is to take place;

(b) a foodstuff or feed is to be prepared or otherwise handled or stored;

(c) washing and/or sanitation is to take place, and/or

(d) a high moisture environment and/or wherein aeration is limited.

Examples of such surfaces include any within an industrial food factory and shelves/benches within a supermarket offering foodstuff. The surface to which the antifungal compound may be applied may be a floor or wall of a building (or a room thereof) or a surface of an article within said room or building. A further example is household use on surfaced against mould.

In a preferred embodiment fungal targets include Candida species, Alternaria species, Aspergillus species, Fusarium species, Cryptococcus species and Trichosporon species.

In a preferred embodiment fungal targets, optionally mould targets include Acremonium species, Alternaria species, Aspergillus species, Cladosporium species, Fusarium species, Mucor species, Penicillium species, Rhizopus species, Stachybotrys species, Trichoderma species, Trichophyton species.

Any of the compounds of the invention as defined above is used against these uses as disclosed herein or in the compositions, preferably the preferred compounds as defined above.

In these uses a method is provided said method comprising suppressing growth of or killing or controlling said fungus, comprising contacting said fungus with the compound of the invention.

### BRIEF DESCRIPTION OF THE FIGURES

FIG. 1. Time-lapse microscopic images (a) of HaCat cells infected by C. albicans in the presence of different concentrations of MICAN and the corresponding fungal growth curves (b) determined from the hyphal area of C. albicans.

FIG. 2. The development strategy for the most effective ICAN derivative.

FIG. 3. (a) Survival curves of mice infected with Candida albicans 3666 isolate. For all groups n=10. (b) Kidney tissue burden of immunosuppressed mice infected by Candida albicans 3666 isolate, two days after infection. (c) six days after infection (p<0.05 (*); p<0.001 (**)).

FIG. 4. (FIG. S1.) ¹H-NMR spectrum of EICAN (400 MHz, CDCl₃, 20° C.)

FIG. 5. (FIG. S2.) ¹³C-NMR spectrum of EICAN (101 MHz, CDCl₃, 20° C.)

FIG. 6. (FIG. S3.) ¹H-NMR spectrum of PICAN (400 MHz, CDCl₃, 20° C.)

FIG. 7. (FIG. S4.) ¹³C-NMR spectrum of PICAN (101 MHz, CDCl₃, 20° C.)

### DEFINITIONS

A “fungus” (plural: fungi) as used herein is a living non-photosynthesizing heterotrophic eukariotic organism belonging to the kingdom of fungi.

Preferably, in fungi as provided herein chitin is present as component of a polymer forming the main structural components of the cell wall. In preferred fungi chitin and glucan are two components of said polymer.

Preferably, a typical fungus comprises hypha (plural: hyphae) formed by cells having a rigid cell wall. The hyphea form filaments forming a network called the mycelium. The mycelium grows by utilizing nutrients from the environment and, upon reaching a certain stage of maturity forms spores.

A “fungal pathogen” is used herein to refer to a fungus which causes damage or is detrimental to a plant or an animal, e.g. is capable of impair said plant or animal.

A “pathogenic fungus” in a wide sense is a fungal pathogen of both plants and animal, preferably of animals.

A “fungal disease” is a condition of a plant or animal which is different from the healthy condition of said plant or animal wherein said difference is caused by a fungus which infects the plant or animal and is thus a fungal pathogen. A variety of environmental and physiological conditions can contribute to the development of fungal diseases. Preferably a fungal disease relates to animals or humans.

“Mycosis” (plural: mycoses) is understood herein as a fungal infection of animals, including humans Alternatively, fungal disease or fungal disease of animals is used. Mycosis may be caused by inhalation of fungal spores or localized colonization of the skin which may initiate infections.

The term “agent” is used in this disclosure to refer to a chemical entity capable of affecting an anti-fungal effect.

An “anti-fungal effect” includes any effect which is against the physiological function of fungi including killing fungi, arresting growth or either sexual or asexual reproduction, or controlling growth or propagation thereof or impairing its cellular functions e.g. its cell membrane or cell wall.

A “composition” or “preparation” as used herein interchangeably, and contains at least one anti-fungal compound of the invention as an agent and a further substance, e.g. a carrier or excipient which has a different utility, e.g. which carries the agent or which stabilizes the agent or maintains it in stable or active form or protects it from environmental effects. Compositions include pharmaceutical and other composition like cleaning agent or preparations to be used in agriculture or plant protection or cosmetics.

A “pharmaceutical composition” or “pharmaceutical preparation” as used herein interchangeably relates to a composition or preparation which contains at least one agent i.e. an anti-fungal compound of the invention and a pharmaceutically-acceptable carrier for use in a method of treatment of an animal e.g. for the treatment or prevention of disease. In the composition, the anti-fungal compound of the invention is present in an amount effective to treat a fungal infection. The term relates to “medicaments” which have to pass a marketing approval procedure defined by the laws of the country or geographic area concerned.

“Therapeutic amount” of a compound of the invention refers to an amount of the compound effective in treating, combating, ameliorating, preventing or improving a condition which is a fungal disease in an animal.

A “subject” is understood herein as an animal or a human being to whom treatment, including prophylactic treatment, with the preparations or compositions of the present invention, is or is to be provided. Preferably the subject is a vertebrate, more preferably a warm-blooded animal, a mammal or a human. Preferably the subject is a patient. A “patient” is a subject who is under medical diagnosis, observation or treatment. The treatment may be preventive or curative. Preferably the patient is a subject having a mycoses or fungal disease.

The term “comprise(s)” or “comprising” or “including” or “containing” are to be construed herein as having a non-exhaustive meaning and allow the addition or involvement of further features or method steps or components to anything which comprises the listed features or method steps or components.

The expression “consisting essentially of” is to be understood herein as consisting of mandatory features or method steps or components listed in a list, whereas allowing to contain additionally other features or method steps or components which do not materially affect the essential characteristics of the same. It is to be understood that “comprise(s)” or “comprising” or “including” or “containing” can be replaced herein by (i.e. limited herein to) “consisting essentially of” or “comprising substantially” or to “consisting of” if so required.

The indefinite articles “a” and “an” are to be construed as including “one or more” unless context defines differently. “One or more” means one or more than one, and may be limited to one, two or three, or one or two, if so required.

### DETAILED DESCRIPTION OF THE INVENTION

Fungi represent highly abundant living organisms worldwide, and wherein about one to two hundred thousand species have been described, much more is estimated. Despite their diversity common features, like chitin in their cell walls and those of their metabolism are well-known.

Combating with damage of fungal origin both in the field of agriculture, hygiene and healthcare is highly important and needed worldwide.

Fungal diseases pose a serious public health problem worldwide, having a great impact in human morbidity and mortality. Immunocompromised individuals are particularly concerned. A further significant threat is present in public area, like swimming pools, gyms and quite importantly healthcare facilities like hospitals.

The present inventors have discovered that 1-amino-5-isocyanonaphthalene (ICAN) and its derivatives exert excellent antifungal activity against a broad range of Candida species. Structure-effect studies based on minimum in vitro inhibitory concentration (MIC) determined for three Candida species, namely C. albicans, C. krusei and C. parapsilosis revealed that for the antifungal effect the naphthalene core, the isocyano- and the amino moieties are all necessary. It was noted that alkylation especially methylation considerably increased the antifungal effect. However, 1,5-diisocyanonaphthalene also showed low MIC (MIC_{PI}=0.3 μg/ml MIC_{TI}=0.6 μg/ml), but its rigid structure and consequently its low solubility in aqueous media may limit its application. Nevertheless, proper formulation and/or substitution with solubility enhancer groups on the naphthalene core may increase its effectiveness. Based on MIC values (MIC_{PI}=0.04−0.3 μg/ml MIC_{TI}=0.6 μg/ml) and its low cytotoxicity 1,1-N-dimethylamino-5-isocyanonaphthalene (DIMICAN) was selected to be tested on clinical Candida isolates. In vitro it was tested using microdilution method against C. albicans (8 isolates), C. krusei (7 isolates), C. tropicalis (7 isolates), C. glabrata (7 isolates) and C. parapsilosis (5 isolates) to get total minimum inhibitory concentration (MIC_{TI}) values of 0.08-1.25 μg/ml. These MIC values are comparable or lower than that of amphotericin B for the same isolates. Amphotericin B (AMB) is an antifungal agent which is effective against a number of medically important fungi. The in vivo applicability of DIMICAN was tested on immunosuppressed mice and control experiments were also conducted with amphotericin B. In the model of acute invasive candidiasis, neutropenic mice were infected intravenously with C. albicans isolate 3666 (3×10⁴ cfu/mouse) to establish an acute infection, and all untreated control animals died within 6 days. DIMICAN (5 mg/kg) prolonged the survival period of animals compared with the survival period of non-treated infected controls, P<0.05; 80 versus 0% survivors at day 7 post infection. Results of fungal burden in kidney revealed that six days after infection AMB and DIMICAN were efficient against C. albicans isolate 3666: p<0.05 (AMB); p<0.001 (DIMICAN). Based on these results and the easy and versatile modification of the ICANs we hope that they have the potential to become an effective clinical lead compound family against pathogenic fungi. As potential lead compounds, ICANs, meet one or more criteria of commercial use, such as low cost, easy preparation and good chemical stability, for example could be used as antimycotic coatings on medical tubes and/or devices. This simple, cheap, nontoxic and easy to prepare compound family would allow not only human treatment, but at the same time could be used as disinfectants and/or coating which is vital against the spread of highly virulent Candida strains.

### Antifungal activity of the ICAN Derivatives of the Invention

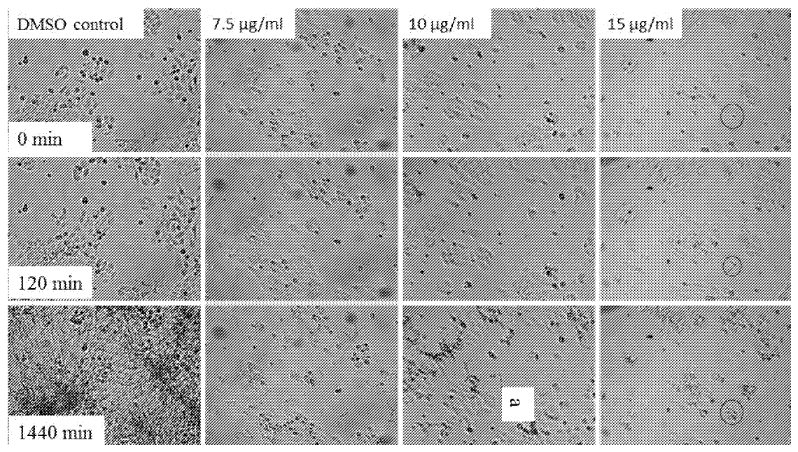

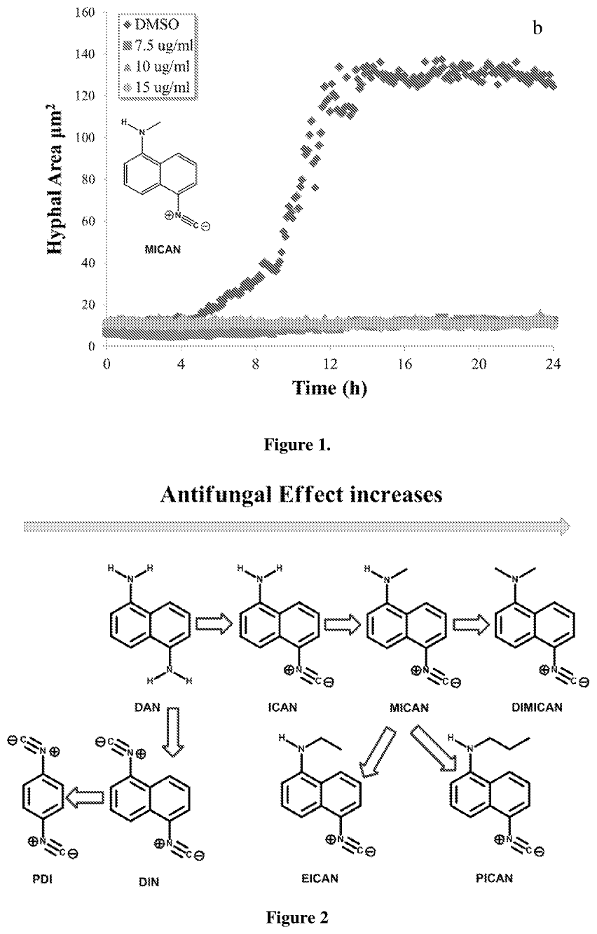

As we mentioned in the Background art, studies have been carried out to successfully utilize 1-N-methylamino-5-isocyanonaphthalene (MICAN) as vital cell dye on different human cell lines [Nagy Z L, 2018]. The compound along with ICAN and 1,1-N-dimethylamino-5-isocyanonaphthalene (DIMICAN) are well tolerated by human cell cultures, their LD₅₀ values were determined (MTT bio assay technique) to be 10 μg/ml for ICAN, 18 μg/ml for MICAN, 13 μg/ml for DIMICAN. Encouraged on these results we wanted to extend their applicability to fungal staining. Since these dyes are nonpolar, they are expected to easily bind to and stain the nonpolar chitin in the cell wall of different fungi species. We tested this assumption on common yeast from a local shop, and much to our surprise after staining, the yeast cells stopped growing and died in a short time. Repeated experiments led to the same conclusion; however, the applied dye concentration was well below the LD₅₀ determined for human cells. We assumed that these compounds may have antifungal effect, therefore C. albicans, one of the most common human pathogenic fungi was selected for further tests. HaCat cell cultures (spontaneously transformed aneuploid immortal keratinocyte cells from adult human skin) were infected with C. albicans 3666 (same isolate as used in animal experiments) and were treated with different concentrations of MICAN dissolved in DMSO. The fungal growth was followed by time lapse imaging for 24 hours. The results are summarized in FIG. 1. As it is evident from FIG. 1a. and 1b, contrary to the untreated (DMSO control) yeast cells, which show the typical yeast growth curve (FIG. 1b), little fungal growth (˜30%) was observed at even as low as 7.5 μg/ml MICAN concentrations, which is well below its LD₅₀ value. It should be noted, however, that in the case of the untreated HaCat cells the fungal growth exceeded 1000% (24 h) calculated from the hyphal area (FIG. 1b, DMSO control). Despite effective fungal growth hindering of MICAN, the treated HaCat cells showed no sign of damage under 24 hours.

It is understood that other fungi are sensitive towards the compounds of the invention. In particular it has been observed that common yeast was highly sensitive. Without being bound by theory the compounds of the invention bind to cell wall elements like chitin as a part of their antifungal action.

### Antifungal Susceptibility Testing—Determination of Structure-Effect Relationship

ICAN has 3 key moieties, namely the amino, naphthalene and isonitrile. To find out which ones of them are essential for the antifungal behavior and to design more efficient derivatives, comparative studies were carried out, where one of the moieties was exchanged or eliminated. The modification/synthetic strategy is summarized in FIG. 2. All ICAN derivatives were tested against C. albicans SC5314, C. krusei ATCC 6258 and C. parapsilosis ATCC 22019. Table 1 summarizes the minimum inhibitory concentrations (MICs) of the ICAN derivatives. The antifungal agents were dissolved in 100% DMSO and diluted further in RPMI-1640 (with L-glutamine but without bicarbonate) and buffered to pH 7.0 with 0.165 M morpholinopropanesulfonic acid (MOPS). MICs were read visually after 48-h incubation at 35° C. using the partial (50%) inhibition (for ICAN derivatives) (PI) and total inhibition (for AMB and ICAN derivatives) (TI) criteria. MICs were determined at least twice.

Based on the data of Table 1 it can be concluded that even the simplest amino-isocyanonaphthalene molecule ICAN has good antifungal effect with isonitrile group is essential MIC_{TI} values between 0.6-5 μg/ml. The starting compound 1,5-diaminonaphthalene (DAN) proved to be completely ineffective against Candida (MIC_{TI}>100 μg/ml), therefore we can conclude that the for the antifungal effect. Next the free amino group of ICAN was converted to isonitrile resulting 1,5-diisocyanonaphthalene (DIN), which showed lower antifungal effect (MIC_{TI}=0.6 μg/ml) than ICAN, probably due to its limited solubility owing to its largely nonpolar and rigid structure. The naphthalene core of DIN was exchanged to phenylene to obtain 1,4-phenylenediisocyanide (PDI). In contrast to DIN, PDI proved to be completely ineffective (MIC_{TI}>100 μg/ml), which means that the naphthalene core is also essential for the antifungal behavior (FIG. 2.). To increase the efficacy of ICAN derivatives, the substitution of the amino group followed. The alkylation of the amino group increases the dipolarity of the molecules, however at the same time it reduces their ability to form H-bonding as an H-bond donor. Monomethylation (MICAN) led to lowered MIC_{TI} values (0.75-1.25 μg/ml), however the use of longer alkyl chains, such as ethyl and propyl (EICAN and PICAN) proved to be not so effective (MIC_{TI}=1.25-2.5 μg/ml and 1.75-2.μg/ml, respectively), may be due to steric hindrance. The lowest MIC values were obtained in the case of DIMICAN (MIC_{PI}≤0.04 μg/ml and MIC_{TI}=0.3-0.6 μg/ml), the dimethylated ICAN derivative. It should be noted that the MICs did not vary significantly for most of the compounds under investigation, only in the case of ICAN was a broader range observed (9-fold variation for ICAN (MIC=0.6-5 μg/ml). For further tests we chose one of the most effective ICAN derivative, DIMICAN which has no cytotoxic effect [Nagy M 2018, Nagy Z L, 2018]. To test the “real life” behavior of DIMICAN it was tested for in vitro susceptibility against clinical isolates collected from patients at the Clinics of the University of Debrecen. Table 2 summarizes the MICs of the new antifungal agent DIMICAN.

TABLE 1
In vitro susceptibilities of C. albicans SC5314, C. parapsilosis
ATCC 22019 , and C. krusei ATCC 6258 to various ICAN derivatives
determined by CLSI broth microdilution method. MICs were read using
the partial (MIC_{PI}) and total inhibition (MIC_{TI}) criteria. [Clinical and
Laboratory Standards Institute. Reference method for broth dilution
antifungal susceptibility testing of yeasts. 2008]. For the full names and
structures of the compounds please refer to FIG. 2 and the EXAMPLES.
The lowest MICs are indicated in bold. For the MIC_{TI} values of AMB for
these Candida strains please refer to Table 2.

| Compound | Candida species | MIC_{PI} (μg/ml) | MIC_{TI} (μg/ml) |
|---|---|---|---|
| ICAN | C. albicans SC5314 | 1.25 | 5 |
| | C. parapsilosis ATCC | 0.3 | 0.6 |
| | 22019 | | |
| | C. krusei ATCC 6258 | 0.6 | 2.5 |
| MICAN | C. albicans SC5314 | 0.3 | 1.25 |
| | C. parapsilosis ATCC | 0.15 | 0.75 |
| | 22019 | | |
| | C. krusei ATCC 6258 | 0.15 | 1.25 |
| EICAN | C. albicans SC5314 | 0.6 | 2.5 |
| | C. parapsilosis ATCC | 0.6 | 1.25 |
| | 22019 | | |
| | C. krusei ATCC 6258 | 0.6 | 1.25 |
| PICAN | C. albicans SC5314 | 0.6 | 2.5 |
| | C. parapsilosis ATCC | 0.6 | 1.75 |
| | 22019 | | |
| | C. krusei ATCC 6258 | 0.15 | 2.5 |
| DIMICAN | C. albicans SC5314 | ≤0.04 | 0.6 |
| | C. parapsilosis ATCC | 0.15 | 0.6 |
| | 22019 | | |
| | C. krusei ATCC 6258 | 0.15 | 0.3 |
| DIN | C. albicans SC5314 | 0.3 | 0.6 |
| | C. parapsilosis ATCC | 0.3 | 0.6 |
| | 22019 | | |
| | C. krusei ATCC 6258 | 0.3 | 0.6 |
| DAN | C. albicans SC5314 | 50 | 100 |
| | C. parapsilosis ATCC | 50 | 100 |
| | 22019 | | |
| | C. krusei ATCC 6258 | 50 | 100 |
| PDI | C. albicans SC5314 | 50 | 100 |
| | C. parapsilosis ATCC | 50 | 100 |
| | 22019 | | |
| | C. krusei ATCC 6258 | 50 | 100 |

TABLE 2
In vitro susceptibilities of Candida isolates to DIMICAN and amphotericin B (AMB),
determined by CLSI broth microdilution method. MICs were determined according to the
CLSI M27-A3 using the partial (MIC_{PI}) and total inhibition (MIC_{TI}) criteria. [22].

| | |
|---|---|
| C. albicans | C. tropicalis |

| | MIC_{PI} | MIC_{TI} | AMB MIC_{TI} | | MIC_{PI} | MIC_{TI} | AMB MIC_{TI} |
|---|---|---|---|---|---|---|---|
| Isolate | (μg/ml) | (μg/ml) | (μg/ml) | Isolate | (μg/ml) | (μg/ml) | (μg/ml) |
| ATCC 10231 | ≤0.04 | 0.3 | 0.25 | ATCC 750 | 0.15 | 1.25 | 0.5 |
| 456 | ≤0.04 | 0.6 | 0.5 | 579 | 0.08 | 0.6 | 0.5 |
| 665 | ≤0.04 | 0.6 | 1 | 622 | 0.08 | 0.3 | 1 |
| 685 | ≤0.04 | 0.6 | 0.25 | 1776 | 0.08 | 0.3 | 1 |
| 720 | ≤0.04 | 0.6 | 0.5 | 2467 | 0.15 | 1.25 | 0.5 |
| 963 | ≤0.04 | 0.6 | 0.5 | 3014 | 0.08 | 0.6 | 0.5 |
| 1544 | ≤0.04 | 0.6 | 0.25 | 5115 | 0.08 | 1.25 | 0.5 |
| 8658 | ≤0.04 | 0.6 | 1 | 5806 | 0.08 | 0.15 | 0.25 |
| 3666 | ≤0.04 | 0.6 | 0.5 | | | | |

| C. glabrata | | | | C. parapsilosis | | | |
|---|---|---|---|---|---|---|---|
| | MIC_{PI} | MIC_{TI} | AMB MIC_{TI} | | MIC_{PI} | MIC_{TI} | AMB MIC_{TI} |
| Isolate | (μg/ml) | (μg/ml) | (μg/ml) | Isolate | (μg/ml) | (μg/ml) | (μg/ml) |
| ATCC 90030 | 0.15 | 0.3 | 0.5 | ATCC 22019 | 0.15 | 0.6 | 0.5 |
| 14408 | ≤0.04 | 0.08 | 0.5 | 4133 | 0.08 | 0.08 | 0.25 |
| 14559 | ≤0.04 | 0.3 | 0.5 | 9613 | 0.15 | 0.15 | 0.25 |
| 22394 | ≤0.04 | 0.3 | 0.5 | 10252 | ≤0.04 | 0.08 | 0.5 |
| 23429 | ≤0.04 | 0.3 | 1 | 18154 | 0.08 | 0.6 | 0.5 |
| 23481 | 0.08 | 1.25 | 1 | 27001 | ≤0.04 | 0.3 | 0.25 |
| 27643 | 0.08 | 0.3 | 0.5 | | | | |
| 28557 | ≤0.04 | 0.6 | 0.5 | | | | |

C. krusei

| | MIC_{PI} | MIC_{TI} | AMB MIC_{TI} |
|---|---|---|---|
| Isolate | (μg/ml) | (μg/ml) | (μg/ml) |
| ATCC 6258 | 0.15 | 0.3 | 1 |
| 749 | 0.08 | 0.08 | 1 |
| 1853 | 0.08 | 0.3 | 1 |
| 4364 | ≤0.04 | 0.15 | 1 |
| 5029 | ≤0.04 | 0.08 | 1 |
| 5072 | 0.08 | 0.3 | 1 |
| 25504 | 0.08 | 0.3 | 0.5 |
| 34987 | ≤0.04 | 0.08 | 1 |

Amphotericin B susceptibility of the isolates was also tested, and their MIC_{TI} values were not higher than 2 μg/ml in each case. DIMICAN MIC_{PI} values were the same or significantly lower compared to MIC_{TI} for all tested five Candida species. The lowest MICs were noticed in case of C. albicans.

### Mouse Model of Infection and Antifungal Therapy

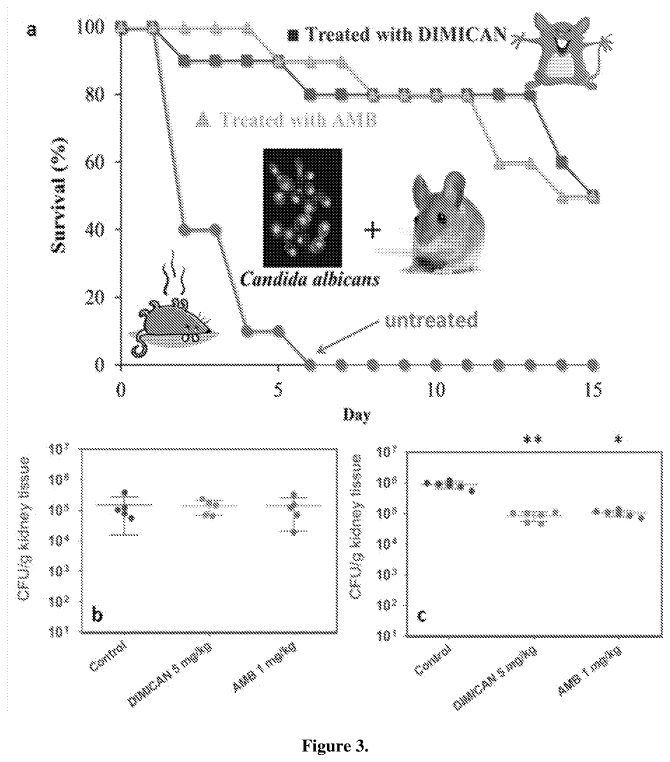

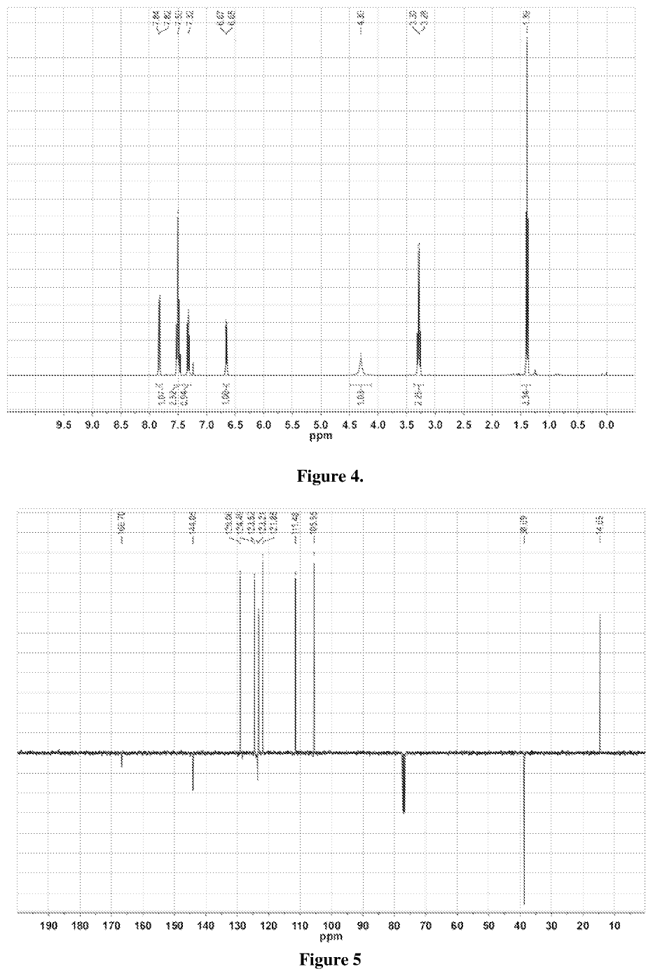

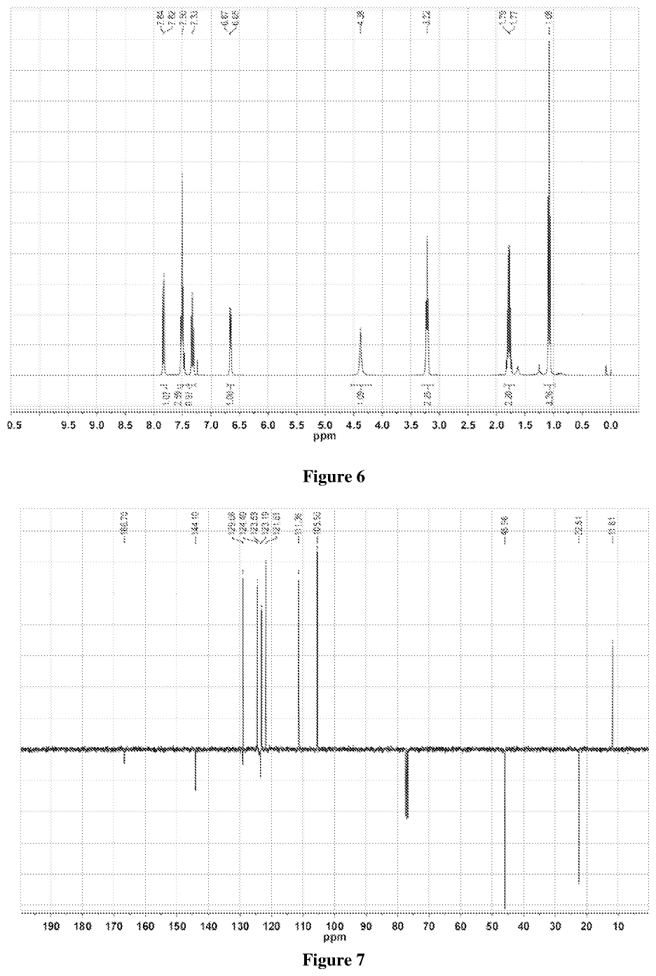

In the model of acute invasive candidiasis, neutropenic mice were infected via the lateral tail vein with C. albicans isolate 3666 (3×10⁴ cfu/mouse) to establish an acute infection. It is well understood that any other fungal pathogens of animals can be used for this purpose. In the example provided herein all untreated control animals died within 6 days. DIMICAN (5 mg/kg) prolonged the survival period of animals compared to non-treated infected controls (P<0.05; 80 versus 0% survivors at day 7 post-infection; FIG. 3a). It should be noted, however, that DIMICAN was administered as intraperitoneal injection which contained DIMICAN as an emulsion in reagent grade olive oil and water. While emulsions are a kind of preferred options in the preparations of the invention, preparing alternative or improved formulations are within the skills of a person skilled in the art.

### Determination of Fungal Burden

During an independent in vivo experiment mice were infected intravenously with C. albicans isolate 3666. The infected mice were euthanized 2 or 6 days after infection (n=5 and 6 mice/time point, respectively), as disclosed herein, to determine the fungal burdens in the kidneys. Kidney burden was analyzed using a Kruskal-Wallis test with Dunn's posttest for multiple comparisons [Foldi R 2012, Kovacs R 2014]. Results of fungal burden experiments are shown on FIGS. 3b and c. Two days after infection AMB and DIMICAN had no effect on fungal load (P>0.05). Six days after infection AMB and DIMICAN treatment resulted in significantly lower fungal burden compared to control: p<0.05 (AMB); p<0.001 (DIMICAN). It is well seen in FIGS. 3a and 3b, that on day 6. the fungal burden increased by an order of magnitude (from −10⁵ to −10⁶) in the case of the untreated mice, while in the case of DIMICAN it remained the same as it was on day 2. The same is valid for AMB. Based on these results it can be concluded that DIMICAN is at least as effective in reducing the fungal burden in the kidney as amphotericin B.

While such experiments are to be done upon the design and preparation of such medicaments it can be advised that the doses of the present compounds may be comparable with that of amphotericin B. It may be that the most effective compounds of the invention may have a lower dose. However, the skilled person will understand that doses may depend on the fungal species, the compound actually applied and the species or specific subject treated. It is also known by person skilled in the art that human equivalent those can be calculated from animal dose in accordance with e.g. FDA guidelines. For example, mouse dose can be divided by 12.3 according to [Guidance for Industry; Estimating the Maximum Safe Starting Dose in Initial Clinical Trials for Therapeutics in Adult Healthy Volunteers U.S. Department of Health and Human Services, Food and Drug Administration Center for Drug Evaluation and Research (CDER) July 2005, Pharmacology and Toxicology]. Thus, a starting human dose in case of DIMICAN may be e.g. about 0.5 mg/kg. Thus, an applicable range of doses may from 0.01 or 0.02 to 50, 30 or 15 mg/kg or from 0.05 to 5 mg/kg, preferably 0.1 to 2 mg/kg. Preferred doses in case of DIMICAN and other preferred compounds may vary between 0.01 mg/kg and 1 mg/kg. More widely, doses may vary between 0.005 mg/kg to 50 mg/kg in the uses of the invention for the preferred compounds of the invention. These ranges can be applied and/or adapted to any antifungal compound of the invention or disclosed herein.

### Methods for the Preparation of the Compounds of the Invention

1,5-diaminonaphthalene (DAN, Aldrich, D21200) was used as received. The synthesis of the ligands: 1-amino-5-isocyanonaphthalene (ICAN), 1-N-methylamino-5-isocyanonaphthalene (MICAN) 1-N,N-dimethylamino-5-isocyanonaphthalene (DIMICAN) and 1,5-diisocyanonaphthalene (DIN) is found in refs.: Racz D et al., 2013 [14]; Nagy M et al., 2014 [17] and in WO2014/184762A1.

1,5-diisocyanonaphthalene (DIN) has a PubChem CID: 55274462.

An exemplary synthesis of synthesis of the ligands: 1-amino-5-isocyanonaphthalene (ICAN) is described in Reference Example 1. Starting from 1-amino-5-isocyanonaphthalene in the presence of a strongly basic environment (e.g. potassium hydroxide) and polar solvent (like dimethyl formamide) the alkyl derivatives of the invention can be prepared by using the respective alkylhalogenide like alkyliodide to obtain the N-alkyl-1-amino-5-isocyanonaphthalene. With the excess of alkyl halogenide used the ratio of the mono- and dialkylated product formed can be controlled. The reaction mixture should be protected from light. Temperature should be controlled, room temperature may be used in particular under the exemplary conditions given herein. Once the products formed the molecules can be obtained by extraction and purification.

### Pharmaceutical Uses

The fungal infection or mycosis may be classified according to the tissue colonized by the fungal pathogen.

### Superficial Mycoses

Superficial mycoses are limited to the outermost layers of the skin or to the mucosal, epidermal and nail surfaces (e.g. mucocutaneous candidiasis).

### Cutaneous Mycoses

Cutaneous mycoses extend deeper into the epidermis, and also include invasive hair and nail diseases. These diseases are restricted to the keratinized layers of the skin, hair, and nails.

### Subcutaneous Mycoses

Subcutaneous mycoses involve the subcutaneous tissues after trauma. These infections are chronic and are difficult to treat and may require surgical interventions such as debridement.

### Systemic Mycoses due to Primary Pathogens

Systemic mycoses due to primary pathogens originate primarily in the lungs and may spread to many organ systems. Organisms that cause systemic mycoses are inherently virulent.

### Systemic Mycoses due to Opportunistic Pathogens

Systemic mycoses due to opportunistic pathogens are infections of patients with immune deficiencies who would otherwise not be infected. Examples of immunocompromised conditions include AIDS, inherited or acquired disorders of granulocytes, inherited or acquired disorders of T lymphocytes, primary immunodeficiencies, alteration of normal flora by antibiotics, immunosuppressive therapy, and metastatic cancer. Examples of opportunistic mycoses include Candidiasis, Cryptococcosis and Aspergillosis.

Fungal pathogens causing mycosis or fungal diseases according to the present invention are selected from e.g. Candida species, Aspergillus species, Cryptococcus species, Histoplasma species, Pneumocystis species, Stachybotrys species

The skilled person understands that these are merely examples and further alternatives exist

### Pharmaceutical Preparations

Carriers, diluents and auxiliary substances are generally pharmaceutically acceptable substances which may be administered without undue toxicity and which, in themselves, will not induce an antifungal effect in the individual receiving a composition comprising any of these substances. Pharmaceutically acceptable carriers include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like.

Once formulated, the composition can be delivered to a subject in vivo using a variety of known routes and techniques. For example, the liquid preparations can be provided as an injectable solution, suspension or emulsion and administered via e.g. parenteral, subcutaneous, intradermal, intramuscular, intravenous or intraperitoneal injection using a conventional needle and syringe, or using a liquid jet injection system. Liquid preparations can also be administered topically to the eyes, to skin, hair or mucosal tissue {e.g. nasal, sublingual, vaginal or rectal), or provided as a finely divided spray suitable for respiratory or pulmonary administration. Other modes of administration include oral administration, suppositories, and active or passive transdermal delivery techniques. In preferred embodiments the antifungal compound is formulated into a composition suitable as a topical cream or lotion, hand-cream, suppository, eye-drop solution, shampoo or conditioner.

The subject in need of the antifungal compound may be any human or animal individual. In preferred embodiments the antifungal compound may be used to prevent infection in subjects at particular risk of acquiring an infection by a fungus and/or to treat infection in subjects at particular risk of being unable to clear a fungal infection without medical intervention.

The invention also relates to a pharmaceutical preparation of formulations for use in a medical condition as defined herein, i.e. a fungal infection or the status of a subject in need of an antifungal treatment, said preparation comprising a compound for use as defined herein and a pharmaceutically acceptable excipient. The pharmaceutical composition is preferably for use in the treatment of a fungal infection (mycosis) selected from the group consisting of superficial infection, cutaneous infection, subcutaneous infection, systemic infection due to primary or opportunistic fungal pathogen. Depending on the site and type of infection the pharmaceutical preparation may be administered in various ways:
topically or by transdermal delivery, in particular in case of superficial, cutaneous and subcutaneous infections,
by inhalation, which is particularly suitable to treat the respiratory system, including the airways, e.g. the lung, nasal and oral cavities etc,
by oral administration, including cases wherein the fungal infection is present in the oral cavities or in the gastrointestinal system e.g. in the gut, and cases when the fungal infection is systemic and resorption of the compound of the invention is required,
transmucosally (including nasal, sublingual, vaginal or rectal delivery), in particular in case of a systemic infection or in case there is an infection at these sites locally and the mucosa is infected,
by injection (such as intradermal, subcutaneous, intramuscular, intravenous, and intraperitoneal), in particular in case of a systemic infection or in case there is an infection at these sites locally.

Special administration routes include e.g. the following.

The present invention concerns oral care compositions including oral washes or rinses, oral gels, toothpaste preparation, and oral spray in a solution comprising the compound of the invention. Typically such preparations comprise an emulsifier.

Topical preparations include preparation with emulsions, liposome preparations etc.

The preparations of the present invention also may comprise stabilizers, e.g. stabilizers against light. In case of solid preparations like tablets light protection may be provided by an appropriate coating. In case of liquid preparations, like injections, washes or lotions, such protection may be provided by appropriate package, bottle or vial which is partly or entirely light non-transmittant.

Preparations against mycoses include those described by [Souza ACO, 2017]. In case of systemic mycoses nanotechnology e.g. nanoparticle preparations may help oral bioavailability of antifungal compounds. In particular, systemic mycoses can be treated by methodology described in this review, including nanoparticle delivery which may facilitate the targeting of organs difficult to reach by traditional methodology.

The invention also relates to a method for treating the fungal infections in humans and animals, as disclosed herein.

The invention also relates to a use of the compound of the invention as an antifungal agent as disclosed herein.

The invention is further illustrated by the examples provided herein. It is to be understood that while certain embodiments are specifically disclosed, those are illustrative and not intended to construe as limitations of the invention.

### EXAMPLES

1,5-diaminonaphthalene (DAN, Aldrich, D21200) was used as received. The synthesis of the ligands: 1-amino-5-isocyanonaphthalene (ICAN), 1-N-methylamino-5-isocyanonaphthalene (MICAN) 1-N,N-dimethylamino-5-isocyanonaphthalene (DIMICAN) and 1,5-diisocyanonaphthalene (DIN) is found in refs.: Racz D et al., 2013 [14]; Nagy Metal., 2014 [17] and in WO2014/184762A1.

### Reference Example 1—Preparation of 1-amino-5-isocyanonaphthalene

### Materials

Acetone, dichloromethane (DCM), hexane, 2-propanol (iPrOH), toluene, (reagent grade, Molar Chemicals, Hungary) were purified by distillation. Acetonitrile (MeCN), tetrahydrofuran (THF), methanol (MeOH), dimethyl -formamide (DMF), dimethyl-sulfoxide (DMSO), pyridine (HPLC grade, VWR, Germany), chloroform, ethyl-acetate (EtOAc), ethanol (EtOH) (reagent grade, Molar Chemicals, Hungary), cyclohexane, 1,4-dioxane (reagent grade, Reanal, Hungary), 1,5-diaminonaphthalene, poly(ethylene glycol) (PEG) and poly(propylene glycol) (PPG) (Sigma-Aldrich, Germany) were used without further purification.

### 1-amino-5-isocyanonaphthalene

A 250 ml three-necked round-bottomed flask, equipped with a condenser, dropping funnel, nitrogen inlet-outlet and magnetic stirrer, was charged with the 1,5-diaminonaphthalene (3.00 g, 19.0 mmol) chloroform (30 ml), toluene (20 ml) and ethanol (10 ml). Potassium-hydroxide (12.0 g, 0,214 mol) dissolved in water (50 cm3) was slowly added to the stirred solution, then refluxed under argon for 12 h. After cooling down, 100 ml acetone was added to the reaction mixture, poured into a separatory funnel, the aqueous phase was removed, and the organic phase was washed with water three times, and then dried over anhydrous magnesium-sulfate. Solvent was removed on rotary evaporator and the residue was purified on a normal-phase silica gel filled column, using methylene chloride as eluent, gave the pure product as pale yellow crystals (0.315 g, 9.9%).

MS (EI) m/z: 168 (M+, 100%; spectra showed 90% similarity to the one of 1-amino-4-cyanonaphthalene in NIST05 library). δH (CDCl3, 400 MHz): 4.24 (2H, s, NH2), 6.87 (1H, dd, J=7.5 and 0.8 Hz, H2), 7.39 (1H, dd, J=7.4 and 8.5 Hz, H3), 7.46 (1H, dd, J=7.5 and 8.3 Hz, H7), 7.57 (1H, d, J=7.3 Hz, H6), 7.63 (1H, d, J=8.4 Hz, H8), 7.88 (1H, d, J=8.6 Hz, H4) ppm. 5C (CDCl3, 100 MHz): 109.6 (C2), 1 12.0 (C4), 120.7 (C8), 121.5 (C7), 121.6 (C9), 122.5 (C6), 126.3 (C3), 126.8 (do), 139.8 (Ci, C5), 162.9 (CNC) ppm. IR vmax (cm KBr disc): 3470, 3430, 3380, 3353 (s, N—H), 2121 (s, NC), 3241, 3064, 3046, 3023, 2923 (m, Ar—H).

### Example 1: General Method for the Synthesis of Alkylated 1-amino-5-isocyanonaphthalenes

A 250 ml round-bottomed flask, equipped with a magnetic stir bar was charged with 1-amino-5-isocyanonaphthalene (1.00 g, 5.94 mmol), potassium hydroxide (0.670 g, 11.9 mmol) and absolute dimethyl formamide freshly distilled over phosphorus pentoxide (100 ml). Alkyliodide (59.4 mmol) was added to the solution, then the flask was flushed with argon and sealed with a rubber septum. Ethyliodide (Sigma-Aldrich, Germany) was used to synthesize N-ethyl-1-amino-5-isocyanonaphthalene (EICAN) (yellow crystals, 370 mg, 32% yield) and N,N-diethyl-1-amino-5-isocyanonaphthalene (diEICAN) (light green waxy compound, 90 mg, 6.8% yield). To synthesize 1-propyl-5-isocyanonaphthalene (PICAN) propyliodide (Sigma-Aldrich, Germany) was used. PICAN (light yellow waxy compound, 440 mg, 35% yield) and N,N-dipropyl-1-amino-5-isocyanonaphthalene diPICAN (green waxy compound, 100 mg, 6.7% yield). With the excess of alkyl iodide used the ratio of the mono- and dialkylated product formed can be controlled. The reaction mixture was stirred at room temperature, protected from light. After 2 days 200 ml methylene chloride and 5% ammonia solution was added, and the solution was extracted 5 times with water, then the organic phase was dried over anhydrous magnesium sulfate. Solvent was removed on a rotary evaporator and the residue was purified on a normal-phase silicagel filled column, using methylene chloride: hexane (1:1) as eluent.

### EICAN (1-N-ethylamino-5-isocyanonaphthalene)

¹H NMR (400 MHz, CDCl₃) δ=7.83 (d, J=8.6 Hz, 1H), 7.50 (q, J=8.6 Hz, 3H), 7.32 (dd, J=8.5, 7.5 Hz, 1H), 6.66 (dd, J=6.8, 1.6 Hz, 1H), 4.30 (s, 1H), 3.29 (q, J=7.1 Hz, 2H), 1.39 (t, J=7.1 Hz, 3H) ppm.
¹³C NMR (101 MHz, CDCl₃) δ=166.70 (quart. Ar—NC), 144.05 (quart. Ar), 129.06 (Ar), 124.49 (Ar), 123.52 (quart. Ar), 123.21 (Ar), 121.85 (Ar), 111.48 (Ar), 105.55 (Ar), 38.69 (Ethyl-CH₂—), 14.65 (Ethyl-CH₃) ppm.

### PICAN (1-N-propylamino-5-isocyanonaphthalene)

¹H NMR (400 MHz, CDCl₃) δ=7.83 (d, J=8.6 Hz, 1H), 7.50 (q, J=8.6 Hz, 3H), 7.32 (dd, J=8.5, 7.5 Hz, 1H), 6.66 (dd, J=6.4, 1.8 Hz, 1H), 4.38 (s, 1H), 3.22 (t, J=7.0 Hz, 2H), 1.85 −1.72 (m, 2H), 1.08 (t, J=7.4 Hz, 3H) ppm.
¹³C NMR (101 MHz, CDCl₃) δ=166.70 (quart. Ar—NC), 144.10 (quart. Ar), 129.06 (Ar), 124.49 (Ar), 123.53 (quart. Ar), 123.19 (Ar), 121.81 (Ar), 111.36 (Ar), 105.50 (Ar), 45.96 (Propyl-CH₂—), 22.51 (Propyl-CH₂—), 11.81 (Propyl-CH₃) ppm.

### Example 2: Biological Studies on Candida albicans 3666

### Time-Lapse Imaging

Non-invasive, near-infrared, extended time-lapse videomicroscopy was used for investigation as previously described [Nagy G, 2010].

The system and protocols for time-lapse scanning (TLS) were composed of:

Incubator. A SANYOMCO18-AC (Wood Dale, Ill., US) CO2 incubator was used with a back-side instrument port. The chamber of the microscope hosted four microscopes.
(a) Microscopes. Olympus (Tokyo, Japan) upright microscopes were modified for inverted usage, and revolver turrets were installed to replace the original illumination. Charge-coupled device (CCD) camera boards were placed under the turrets, using the monocular adapter lower parts of Olympus Tokyo as housing. Specimen tables were unmodified, but the slide orientation mechanisms were removed. Ocular sockets were used for illuminators.
(b) Illumination. Diodes (LED: 5 mm diameter; 1.2 V and 50 mA, driven at 5 V using a serial 82 Ohm resistor) emitting light at 940 nm were used to illuminate cells at minimized heat and phototoxicity. Longer wavelength offered a deeper penetration (up to 3-mm thickness) and less light dispersion through the culture medium and the wall of the T-flask. The theoretical limit of resolution under our conditions using a 940-nm wavelength at 1.25 numerical aperture was 1.88 nm based on the Abbe equation. The original 5 mm spherical light-emitting diode (LED) was used as a condenser for a better reproducibility of the setup. Illuminators were centered and glued to the ocular tubes of the microscope. The distance between the upper surface of the T-25 culture flask and the spherical ballhead of the LED was 120 mm. Illumination of cells was limited to the image acquisition periods (˜5 sec/timepoint).
(c) Microscope objectives. Carl Zeiss (Jena, Germany) plan achromatic objectives (*10/0.25 NA) were used to enable a broad field of view to be imaged.
(d) Cameras. Custom-modified 2 megapixel UVC USB 2.0 camera boards (Asus Computer International, Fremont, Calif., USA) were modified by removing the camera housing, objectives, and infrared-cut filters. Status indicator LEDs and attached resistors were desoldered, and driver circuit terminals were elongated with wires for near infrared illumination.

### Digital Image Analysis

Image analysis plug-ins were developed by a Java-based image processing software, The ImageJ v1.39d (US National Institutes of Health) program enabled the quantitative analysis of fungi. The software is requesting a set of data for the analysis from the user, along with the directory in which the bank of images to be analysed are contained. Image processing and quantitative image analysis were carried out as previously described [Nagy G, 2014].

### Image Processing

Image pre-processing begun with the filtering of images to compensate the low-frequency, uneven illumination, which could have resulted in shading and a non-uniform, flickering background. This necessitated a segmentation process since grey levels exist within images that are common to both objects and background. The selection of a single threshold level to separate objects and background was therefore not feasible. Applying a single grey-level threshold to such images would have resulted in a high level of artefacts.

Median Bypass Filter. Variations in the background grey level could be corrected by handling an image as a 2D signal and subjecting it to high-pass filtering, which is implemented by using ImageJ's inbuilt “Bandpass Filter” function. This removes image noises by Gaussian filtering in a Fourier space. The radius of the filter was set to 40 μm (40 pixels for images of hyphae, 200 pixels for yeast cells). The radius of the width of filter is large relative to the size of a typical hypha (approximately 2-4 μm). Any high-frequency speckles of background noise are subsequently removed by median filtering

Binary image formation. The images were then divided into three, 8-bit grey-scale images, representing the three primary colour components (red, green, and blue, RGB). The red component exhibits the greatest contrast for images of the lactophenol cotton blue-stained fungal samples. These components were retained for image processing; the green and blue components discarded. A grey-level threshold, calculated by ImageJ using the iso-data algorithm, was then applied, resulting in a binary image.

Temporal distinction of hyphae. The next stage of the routine depended on whether yeast cells or hyphae were the primary object of study. It was unlikely that the experimental setup viable yeast cells and hyphae were present in the same image. Preliminary results indicated that most yeast cells differentiated approximately 8 h after inoculation. Yeast cells present in a sample taken after this time were considered to be non-viable and regarded by the system as artefacts.

Distinction of yeast cells. The “Watershed” function was used to separate any touching objects, primarily yeast cells. The command calculated the Euclidean Long Distance Map (EDM) first then finds the final Eucledian Eroded Points (UEP). It then extended the individual UEPs up to the edge of the particle or to another UEP region. Water jet segmentation was best suited for smooth, round or convex objects that did not overlap.

### Example 3: Clinical Isolates and Antifungal Susceptibility Testing

The tested C. albicans, C. tropicalis, C. parapsilosis, C. glabrata and C. krusei ATCC strains and clinical isolates are listed in Table 2. MICs were determined according to the CLSI M27-A3 [22]. Antifungal agents were dissolved in 100% DMSO and diluted further in RPMI-1640 (with L-glutamine but without bicarbonate) (Sigma, Budapest, Hungary), and buffered to pH 7.0 with 0.165 M morpholinopropanesulfonic acid (MOPS; Sigma, Budapest, Hungary). The final concentration ranges used for AMB and ICAN derivatives were 0.125 to 4 μg/ml and 0.04 to 20 μg/ml, respectively. Yeast cell suspensions were prepared in RPMI 1640 medium and were adjusted to give a final inoculum concentration of 0.5×10³ to 2.5×10³ cells/ml. MICs were read visually after 48-h incubation at 35° C. using the partial (50%) inhibition (for ICAN derivatives) (PI) and total inhibition (for AMB and ICAN derivatives) (TI) criteria. MICs were determined at least twice. For amphotericin B isolates with MIC values of ≤2 mg/L were considered wild-types. [Pfaller M A, 2012]

### Murine Model of Infection

We used 10-12 week-old female BALB/c mice for all experiments. Mice were immunosuppressed using 150 mg/kg intraperitoneal (ip) cyclophosphamide dose (4 days prior to infection) and 100 mg/kg ip cyclophosphamide dose (1 day prior to infection; 2 and 5 days after infection) [Spellberg B, 2005]. All mice received ceftazidime (5 mg/day subcutaneously) from days 0 to 6 after infection. Mice were given food and water ad libitum and were monitored daily. The animals were maintained in accordance with the Guidelines for the Care and Use of Laboratory Animals, and the experiments were approved by the Animal Care Committee of the University of Debrecen, Debrecen, Hungary (permission no. 12/2014).

Mice were infected via the lateral tail vein with C. albicans 3666 bloodstream isolate. In the lethality (10 mice/group) and fungal tissue burden experiments (6 mice/group) the infectious doses were 3×10⁴ and 10⁴ cfu/mouse, respectively. These doses led to 100% and 0% mortality, respectively by day 6 after infection among untreated mice. Inoculum density was confirmed by plating serial dilutions onto Sabouraud dextrose agar (SDA) plates.

### Antifungal Therapy

AMB (Fungizone, commercial preparation) and DIMICAN treatment in a 0.5-ml bolus was started 1 h after infection. The doses were 1 mg AMB and 5 mg DIMICAN (in olive oil/water (o/w) emulsion) per kg body weight. All antifungals were administered intraperitoneally once daily for 5 consecutive days.

### Determination of Fungal Burden

Infected mice were euthanized 2 or 6 days after infection (n=6 mice/time point) to determine the fungal burden in the kidney. The organs were aseptically removed, weighed, homogenized in sterile saline, serially diluted 10-fold in sterile saline. Aliquots of 0.1 ml of the undiluted and diluted homogenates were plated onto SDA, and the colony count was determined after 48 h. Colony-forming units (CFUs) were determined after 48 h of incubation at 37° C. and results were expressed as cfus/g tissue [Spellberg B, 2005]. The lower limit of detection was 50 cfu/g tissue [Foldi R 2012, Kovacs R 2014].

### Statistical Test

Survival effects were analyzed by the Kaplan—Meier log-rank test. Kidney burden was analysed using a Kruskal—Wallis test with Dunn's posttest for multiple comparisons [Foldi R 2012, Kovacs R 2014].

### INDUSTRUAL APPLICABILITY

The invention present relates to ICAN-derivative compounds as defined herein, their use as antifungal agents and any further antifungal uses, pharmaceutical and non-pharmaceutical antifungal preparations as well as said ICAN-derivative compounds and pharmaceutical preparations for use in the treatment of a subject in need of antifungal medication. Moreover, the invention present relates to methods of treatment of fungal diseases by using the ICAN-derivative compounds as well as the pharmaceutical antifungal preparations of the invention.

### REFERENCES

[1] Pfaller MA, Diekema DJ. Epidemiology of invasive candidiasis: a persistent public health problem. Clin Microbio) Rev. 2007 January; 20 (1):133-6:3.
[2] Bongomin F, Gago S, Oladele R O, Denning D W. Global and Multi-National Prevalence of Fungal Diseases-Estimate Precision. J Fungi (Basel). 2017; 3 (4):57. Published 2017 Oct. 18. doi:10.3390/jof3040057
[3] Garcia-Solache M A, Casadevall A. Global warming will bring new fungal diseases for mammals. MBio. 2010; 1 (1):e00061-10. Published 2010 May 18. doi:10.1128/mBio.00061-10
[4] Bondaryk M, Kurzatkowski W, Staniszewska M. Antifungal agents commonly used in the superficial and mucosal candidiasis treatment: mode of action and resistance development. Postepy Dermatol Alergol. 2013; 30 (5):293-301.doi:10.5114/pdia.2013.
[5] Odds F C, Hanson M F, Davidson A D, Jacobsen M D, Wright P, Whyte J A, Gow N A and Jones B L. 2007. One year prospective survey of Candida bloodstream infections in Scotland. J MedMicrobiol 56: 1066-1075.
[6] Cauchie M, Desmet S, Lagrou K Candida and its dual lifestyle as a commensal and a pathogen. Res Microbiol. 2017 November-December; 168 (9-10):802-810. doi: 10.1016/j.resmic.2017.02.005. Epub 2017 Mar. 2.
[7] Lortholary O, Renaudat C, Sitbon K, Madec Y, Denoeud-Ndam L, Wolff M, Fontanet A, Bretagne S, Dromer F; French Mycosis Study Group. Worrisome trends in incidence and mortality of candidemia in intensive care units (Paris area, 2002-2010). Intensive Care Med. 2014 September; 40 (9):1303-12. doi: 10.1007/s00134-014-3408-3. Epub 2014 Aug. 6.
[8] Hirano R, Sakamoto Y, Kudo K, Ohnishi M. Retrospective analysis of mortality and Candida isolates of 75 patients with candidemia: a single hospital experience. Infect Drug Resist. 2015; 8:199-205. Published 2015 Jul. 8. doi:10.2147/IDR.S80677
[9] Yapar N. Epidemiology and risk factors for invasive candidiasis. Ther Clin Risk Manag. 2014; 10:95-105. Published 2014 Feb. 13. doi:10.2147/TCRM.S40160
[10] Ben-Ami R. Treatment of Invasive Candidiasis: A Narrative Review. J Fungi (Basel). 2018; 4 (3):97. Published 2018 Aug. 16. doi:10.3390/jof4030097
[11] Perfect J R. The antifungal pipeline: a reality check. Nat Rev Drug Discov. 2017; 16 (9):603-616. doi:10.1038/nrd.2017.46
[12] Cortegiani A, Misseri G, Fasciana T, Giammanco A, Giarratano A, Chowdhary A. Epidemiology, clinical characteristics, resistance, and treatment of infections by Candida auris. J Intensive Care. 2018; 6:69. Published 2018 Oct. 29. doi:10.1186/s40560-018-0342-4
[13] Krysan D J. The unmet clinical need of novel antifungal drugs. Virulence. 2017; 8 (2):135-137. Published 2017 Jan. 17. doi:10.1080/21505594.2016.1276692
[14] Racz D, Nagy M, Mandi A, Zsuga M and Keki S. 2013. Solvatochromic properties of a new isocyanonaphthalene based fluorophore. J Photoc Photobio. A, 270: 19-27. [1]
[15] Nagy M, Racz D, Nagy Z L, Feher P P, Kalmar J, Fabian I, Kiss A, Zsuga M and Keki K. 2018. Solvatochromic isocyanonaphthalene dyes as ligands for silver (I) complexes, their applicability in silver (I) detection and background reduction in biolabelling Sens Actuators B Chem, 255, 2555-2567. a
[16] Nagy, Miklós; Kovács, Sándor Lajos; Nagy, Tibor; Rácz, Dávid; Zsuga, Miklós; Kéki, Sándor. Isocyanonaphthalenes as extremely low molecular weight, selective, ratiometric fluorescent probes for Mercury(II) Talanta Volume 201, 15 Aug. 2019, Pages 165-173
[17] Nagy M, Rácz D, Lázár L, Purgel M, Ditrói T, Zsuga M, et al. Solvatochromic Study of Highly Fluorescent Alkylated Isocyanonaphthalenes, Their π-Stacking, Hydrogen-Bonding Complexation, and Quenching with Pyridine. Chemphyschem. 2014; 15 (16):3614-25.
[18] Nagy M, Rácz D, Nagy Z L, Nagy T, Fehér P P, Purgel M, et al. An acrylated isocyanonaphthalene based solvatochromic click reagent: Optical and biolabeling properties and quantum chemical modeling. Dyes Pigm. 2016; 133:445-57.
[19] Nagy M, Keki S, Racz D, Mathur J, Vereb G, Garda T, M-Hamvas M, Chaumont F, Boka K, Boddi B, Freytag C, Vasas G and Mathe C. 2018. Novel fluorochromes label tonoplast in living plant cells and reveal changes in vacuolar organization after treatment with protein phosphatase inhibitors, PROTOPLASMA, DOI: 10.1007/s00709-017-1190-0 b
[20] Nagy Z L, Nagy M, Kiss A, Racz D, Barna B, Konczol P, Bank C, Bacs Z, Keki S, Banfalvi G and Szeman-Nagy G. 2018. MICAN, a new fluorophore for vital and non-vital staining of human cells, Toxicology in Vitro, DOI: 10.1016/j.tiv.2018.01.012
[21] Sándor Lajos Kovács, Miklós Nagy, Péter Pál Fehér, Miklós Zsugaand Sándor Kéki, Effect of the Substitution Position on the Electronicand Solvatochromic Properties of Isocyanoaminonaphthalene (ICAN) Fluorophores Molecules 2019, 24, 2434; doi:10.3390/molecules24132434www.mdpi.com/journal/molecules
[22] Clinical and Laboratory Standards Institute. Reference method for broth dilution antifungal susceptibility testing of yeasts. Approved standard, ed 3. CLSI document M27-A3. Wayne, Clinical and Laboratory Standards Institute, 2008.
[23] Foldi R, Kovacs R, Gesztelyi R, Kardos G, Berenyi R, Juhasz B, Szilagyi J, Mozes J, Majoros L. 2012. Comparison of in vitro and in vivo efficacy of caspofungin against Candida parapsilosis, C. orthopsilosis, C. metapsilosisand C. albicans. Mycopathol. 174, 311-318.
[24] Kovacs R, Gesztelyi R, Berenyi R, Doman M, Kardos G, Juhasz B, Majoros L. 2014. Killing rates exerted by caspofungin in 50% serum and its correlation with in vivo efficacy in a neutropenic murine model against Candida krusei and Candida inconspicua. J Med Microbiol. 63: 186-194
[25] Kouznetsov V V., Susan. A. Zacchino, Maximiliano Sortino, Leonor Y. Vargas Méndez, Mahabir P. Gupta, Cytotoxic and Antifungal Activities of Diverse α-Naphthylamine Derivatives. SciPharm 2012, 80: 867-877
[26] Dobnica L. et al. Antifungal Activity of Isothiocyanates and Related Compounds Applied Microbiology, April 1968, p. 582-587.
[27] Pfaller M A, Espinel-Ingroff A, Canton E et al. Wild-type MIC distributions and epidemiological cutoff values for amphotericin B, flucytosine, and itraconazole and Candida spp. as determined by CLSI broth microdilution. J Clin Microbiol 2012; 50:2040-6.
[28] Spellberg B, Ibrahim A S, Edwards J E Jr, Filler S G. 2005. Mice with disseminated candidiasis die of progressive sepsis. J Infect Dis. 192: 336-343.
[29] Nagy G, Pinter G, Kohut G, Adam A L, Trencsenyi G, Hornok L, Banfalvi G, 2010 Time-lapse analysis of cell death in mammalian and fungal cells. DNA Cell Biol 29 (5):249-259.
[30] Nagy, Gabor, Grant W Hennig, Katalin Petrenyi, Laszlo Kovacs, Istvan Pocsi, Viktor Dombradi, Gaspar Banfalvi, Time-lapse video microscopy and image analysis of adherence and growth patterns of Candida albicans strains, Applied Microbiology and Biotechnology, June 2014, Volume 98, Issue 11, pp 5185-5194
[31] Souza, A C O and Amaral A C 2017 Antifungal Therapy for Systemic Mycosis and the Nanobiotechnology Era: Improving Efficacy, Biodistribution and Toxicity. Front Microbiol. 2017; 8: 336.

1. A method for treatment of a fungal infection in a subject having a fungal disease caused by a fungal pathogen wherein a compound having general formula (I) 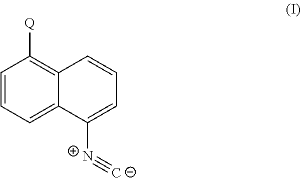
wherein Q is selected from the group consisting of cyano (—NC), nitro (—NO2), nitroso (—NO), hydroxylamino (—NHOH), isothiocyano (—NCS), or an amino having the general formula (i): 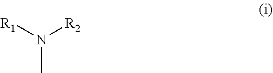
wherein:
R₁ is H or a C₁-C₄ alkyl, a C₂-C₄ alkenyl or a C₂-C₄ alkynyl,
R₂ is H or a C₁-C₄ alkyl, a C₂-C₄ alkenyl or a C₂-C₄ alkynyl,
or R₁ and R₂ together form a C₃-C₈ cykloalkyl or a C₅-C₈ cykloalkenyl,
or a pharmaceutically acceptable salt thereof,
is administered in an amount sufficient to reduce the level of the fungal pathogen in the subject or to prevent proliferation or suppressing growth thereof.

2. The compound for use method for treatment according to claim 1 wherein said compound has formula (II) 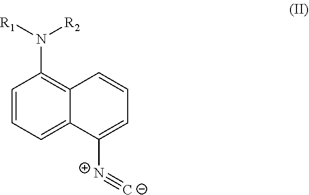
wherein in the formula
R₁ and R₂ is, independently, selected from the group consisting of H, methyl, ethyl and propyl, with the proviso that at least one of R1 and R2 is different from H,
or R₁ and R₂ together form a cyclobutyl, cyclopentyl, cyclohexyl or cycloheptyl.

3. The method for treatment according to claim 2,
said compound being selected from
1-amino-5-isocyanonaphthalene (ICAN)
N-methyl-1-amino-5-isocyanonaphthalene (MICAN)
N-ethyl-1-amino-5-isocyanonaphthalene (EICAN)
N-(prop-1-yl)-1-amino-5-isocyanonaphthalene (PICAN)
N,N-dimethyl-1-amino-5-isocyanonaphthalene (DIMICAN)
1,5-diisocyanonaphthalene (DIN).

4. The method for treatment according to claim 1,
wherein the fungal pathogen is selected from Candida, Aspergillus, Alternaria, Fusarium, Cryptococcus, Histoplasma, Pneumocystis, Stachybotrys and Trichosporon, preferably Candida, Aspergillus Cryptococcus and Trichosporon.

5. The for treatment according to claim 4 wherein the fungal pathogen is Candida and the fungal disease is candidiasis.

6. The method for treatment according to claim 1 wherein the subject is an animal, preferably a mammal, more preferably a human.

7. The method for treatment according to claim 1 wherein said compound is present in a pharmaceutical preparation comprising a pharmaceutically acceptable excipient, carrier or diluent, said preparation being formulated for antifungal administration.

8. The method for treatment according to claim 7 wherein the fungal disease is a systemic fungal infection and said pharmaceutical preparation is formulated for systemic administration, preferably for oral administration or administration by injection.

9. The method for treatment according to claim 7 wherein the fungal disease is a local or multilocal fungal infection, e.g. a cutaneous, subcutaneous or mucosal infection and said pharmaceutical preparation is formulated for topical administration or optionally for systemic administration.

10. A method for use of the compound having general formula (I) 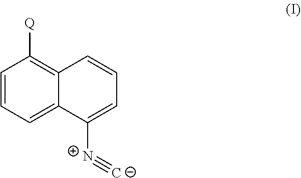
wherein Q is selected from the group consisting of
cyano (—NC), nitro (—NO2), nitroso (—NO), hydroxylamino (—NHOH), isothiocyano (—NCS), or an amino having the general formula (i): 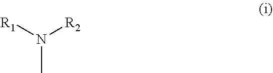
wherein
R₁ is H or a C₁-C₄ alkyl, a C₂-C₄ alkenyl or a C₂-C₄ alkynyl,
R₂ is H or a C₁-C₄ alkyl, a C₂-C₄ alkenyl or a C₂-C₄ alkynyl,
or R₁ and R₂ together form a C₃-C₈ cykloalkyl or a C₅-C₈ cykloalkenyl,
to prevent the growth of and/or to kill a fungus,
said method comprising
contacting said fungus with said compound.

11. A pharmaceutical composition for use in a method of preventing or treating a fungal disease in an animal, said composition comprising a compound having general formula (I) 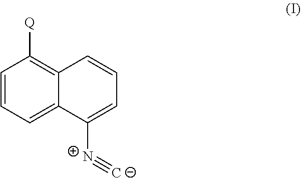
wherein Q is selected from the group consisting of
cyano ( NC), nitro (—NO2), nitroso (—NO), hydroxylamino (—NHOH), isothiocyano (—NCS), or an amino having the general formula (i): 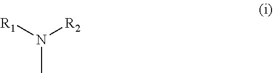
wherein
R₁ is H or a C₁-C₄ alkyl, a C₂-C₄ alkenyl or a C₂-C₄ alkynyl,
R₂ is H or a C₁-C₄ alkyl, a C₂-C₄ alkenyl or a C₂-C₄ alkynyl,
or R₁ and R₂ together form a C₃-C₈ cykloalkyl or a C₅-C₈ cykloalkenyl,
and a pharmaceutically acceptable excipient, carrier or diluents.

12. The pharmaceutical composition of claim 11, wherein the animal is a vertebrate animal, preferably a fish, amphibian, reptilian, bird or mammal, in particular a mammal, highly preferably a human subject.

13. The pharmaceutical composition of claim 11 for use in an organ of the body which provides barrier function, preferably said organ being selected from the group consisting of the skin, respiratory tract, gastrointestinal tract and the genital-urinary tract.

14. The pharmaceutical composition of claim 11 or use in the treatment of a fungal infection or mycosis selected from the group consisting of superficial mycoses, cutaneous mycoses, subcutaneous mycoses and systemic mycoses including systemic mycoses due to primary pathogens and systemic mycoses due to opportunistic pathogens.

15. The pharmaceutical composition for use of claim 11 said composition being selected from the group consisting of topical preparations e.g. preparations by transdermal delivery, in particular in case of superficial, cutaneous and subcutaneous infections, inhalable preparations, oral preparations, including solid preparations and oral washes, transmucosal preparations including nasal, sublingual, vaginal or rectal delivery preparations and injections, such as intradermal, subcutaneous, intramuscular, intravenous, and intraperitoneal injections.

16. The method according to claim 10, wherein said compound has formula (II) 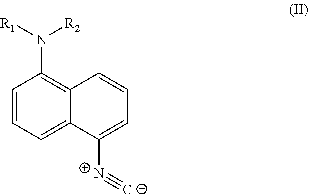
wherein in the formula
R₁ and R₂ is, independently, selected from the group consisting of H, methyl, ethyl and propyl, with the proviso that at least one of R1 and R2 is different from H,
or R₁ and R₂ together form a cyclobutyl, cyclopentyl, cyclohexyl or cycloheptyl.

17. The method according to claim 16, said compound being selected from
1-amino-5-isocyanonaphthalene (ICAN)
N-methyl-1-amino-5-isocyanonaphthalene (MICAN)
N-ethyl-1-amino-5-isocyanonaphthalene (EICAN)
N-(prop-1-yl)-1-amino-5-isocyanonaphthalene (PICAN)
N,N-dimethyl-1-amino-5-isocyanonaphthalene (DIMICAN)
1,5-diisocyanonaphthalene (DIN).

18. The method according to claim 10, wherein said compound is used as an antifungal agent, a fungicide, a disinfectant or an antifungal preservative.

19. The method according to claim 10,
wherein the fungus is selected from
Candida species, Alternaria species, Aspergillus species, Fusarium species, Cryptococcus species and Trichosporon species; and/or
wherein the fungus is a mold selected from Acremonium species, Alternaria species, Aspergillus species, Cladosporium species, Fusarium species, Mucor species, Penicillium species, Rhizopus species, Stachybotrys species, Trichoderma species, Trichophyton species.

20. The pharmaceutical composition according to claim 11 wherein said compound has formula (II) 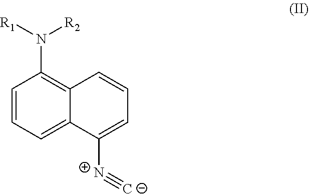
wherein in the formula
R₁ and R₂ is, independently, selected from the group consisting of H, methyl, ethyl and propyl, with the proviso that at least one of R1 and R2 is different from H,
or R₁ and R₂ together form a cyclobutyl, cyclopentyl, cyclohexyl or cycloheptyl.

21. The pharmaceutical composition according to claim 11 said compound being selected from
1-amino-5-isocyanonaphthalene (ICAN)
N-methyl-1-amino-5-isocyanonaphthalene (MICAN)
N-ethyl-1-amino-5-isocyanonaphthalene (EICAN)
N-(prop-1-yl)-1-amino-5-isocyanonaphthalene (PICAN)
N,N-dimethyl-1-amino-5-isocyanonaphthalene (DIMICAN)
1,5-diisocyanonaphthalene (DIN).
